(19) United States
(12) Patent Application Publication
(10) Pub. No.: US 2022/0183274 A1
(43) Pub. Date: Jun. 16, 2022

(54) METHOD TO PACKAGE A TISSUE MATRIX TO BE REGENERATED

(71) Applicant: THERACELL CONSULTING SPRL, Lasne (BE)

(72) Inventor: Denis DUFRANE, Lasne (BE)

(73) Assignee: THERACELL CONSULTING SPRL, Lasne (BE)

(21) Appl. No.: 17/600,074

(22) PCT Filed: Apr. 4, 2019
(86) PCT No.: PCT/EP2019/058538

§ 371 Date: Sep. 29, 2021

(51) Int. Cl. A01N 1/02; A61L 27/36; A61L 27/38

(52) U.S. Cl. CPC A01N 1/0242; A01N 1/0263; A01N 1/0289; C12N 5/0697; A61L 27/3691; A61L 27/3834; A61L 2400/06; A61L 27/3604

(57) ABSTRACT

Biological tissue is packaged to be regenerated before grafting in a vial sealed under vacuum and comprising a biological tissue matrix. A method for producing said vial includes placing a treated ex-vivo tissue sample in an open rigid vial and placing the vial in a lyophilizer. A lyophilization process is performed under vacuum to convert the treated ex-vivo tissue sample into a biological tissue matrix. The vial is hermetically sealed with closing means, inside the lyophilizer under vacuum. The sealed vial is then removed from the lyophilizer.

The invention relates to the field of packaging a biological tissue to be regenerated before grafting.

Biological tissues used for autologous, allogenic or xenogeneic grafts or transplants can be obtained from tissues extracted from donors (of human or animal origin) during surgical interventions, like for example femoral bones during placement of a hip prosthesis or soft tissues like human fascia lata. The extracted biological tissue undergoes a number of treatment steps to be cleaned from any living element (cells, bacteria, . . . ) in order to produce a treated ex-vivo tissue sample with further undergoes dehydration to give a biological tissue matrix, which can then be conditioned to be ready for use. The use of the tissue matrix implies the regeneration into a regenerated tissue by placing the tissue matrix in presence of a biological fluid.

For example, Dufrane et al., Biomaterials 23 (2002) 2979-2988 disclose such a process to treat physically and chemically fascia lata. The treatment steps are typically centrifugating the extracted tissue to eliminate most of the blood and/or fat it contains; cutting the tissue in pieces of the desired size, treating chemically the tissue to eliminate any trace of cells, virus of bacteria. After the treatment, the treated tissue is lyophilized and finally packaging as a tissue matrix.

In order to meet the standards of safety regulations, the above process need to be performed in sterile and/or classified conditions (i.e. meeting regulatory standards). The treatment is time consuming when performed by an operator, expansive and must lead to a product which can be stored for a long time.

Usually, pieces of biological tissue matrixes are conditioned in a dry form, in soft packages, in order to remain dry as long as possible, or in hard permeable packages. The treated ex-vivo tissue sample is lyophilized to a maximum of 6% humidity content, removed from lyophilizer and the resulting tissue matrix is conditioned. However, the mere step of removing the tissue matrix from the lyophilizer before packaging induces take up of ambient water thereby limiting the shelf-life of the product, and a risk of contamination of the tissue.

When a medical professional need to use a biological tissue matrix, conditioned as above, he opens the package containing the tissue matrix and regenerates it to a biological tissue, i.e. he rehydrates the tissue with fluids from the receiving patient. This ensures successful graft and minimizes the risk of graft rejection.

This step involves manipulation of the tissue matrix which involves a risk of contamination.

The applicant therefore judges necessary to propose a new method to package tissue matrix that reduces the risk of contamination before grating and improves its shelf life, as well as a packaged biological tissue to be regenerated obtained by this method.

### Solution of the Invention

To this purpose, it is proposed a method for producing a vial, under vacuum and comprising a biological tissue matrix to be regenerated for grafting comprising the steps of:
placing a treated ex-vivo tissue sample in an open rigid vial;
placing the vial in a lyophilizer;
running a lyophilization process under vacuum to convert the treated ex-vivo tissue sample into a biological tissue matrix;
hermetically sealing the vial with closing means, inside the lyophilizer under vacuum, and
removing the sealed vial from the lyophilizer.

Preferably, before running the lyophilisation process, before or after placing the vial in the lyophilizer, the method further comprises the step of:
pre-positioning the closing means onto the vial in a manner enabling fluid communication through the opening of the vial, i.e. between the inside and the outside of the vial.

Vacuum is to be understood as low pressure as is generally encountered in standard lyophilizer. For example a pressure below 1 mbar is reached, preferably a pressure below 50 pbar and still preferably below 10 pbar.

This method enables to obtain a vial, for clinical use, sealed with closing means under vacuum and comprising a biological tissue matrix to be regenerated for grafting. Regeneration is usually performed by adding cells in suspension in blood, bone marrow, lipoaspirate, or with a growth factors suspension, a culture media with stem cells or any other bioactive agents. The closing means are preferably resealable.

By “clinical use”, one should understand that the standards of the materials and manufacturing conditions should match official regulations. For example, materials are preferably of the medical or pharmaceutical grade, the production/manufacture is performed in a sterile environment of sterilization steps are included.

By “biological tissue”, it is referred to a tissue extracted from donors (of human or animal origin) during surgical interventions, like for example femoral bones during placement of a hip prosthesis or soft tissues like human fascia lata.

By “treated ex-vivo tissue sample”, it is referred to the extracted biological tissue after it has undergone a number of treatment steps (physical like for example centrifugation, or chemical, like for example antibacterial treatment) in order to be cleaned from any living element (cells, bacteria, . . . ) usually present in a biological tissue.

By “biological tissue matrix” or “tissue matrix”, it is referred to the ex-vivo tissue sample after it has undergone a dehydration step, typically by lyophilization.

By “regenerated tissue”, it is referred to the biological tissue matrix after it has been placed in presence of a biological fluid containing elements suitable to reconstitute the matrix into a proper tissue which is ready to be grafted.

The invention also relates to the use of the vial sealed under vacuum comprising a biological tissue matrix (to be regenerated for grafting) for producing a regenerated tissue.

The invention also relates to the method to regenerate the biological tissue matrix, packaged in the vial sealed under vacuum, to a biological tissue, comprising the steps of:
introducing in the vial a regenerating fluid to regenerate the tissue matrix to a biological tissue.

The vial can be, for example, a vial or flask or bottle in glass or in other material, like a polymer or plastic, having a thickness and composition suitable to bear the difference of pressure between the inside and the outside of the vial. It should also be suitable for contact with the biological tissue matrix, meaning it should not release any chemical into the biological matrix. The vial is preferably sterile and/or of pharmaceutical, surgical, clinical or veterinary grade.

The closing means can be any suitable stopper or lid able to ensure that vacuum is maintained in the vial for a long period of time and that humidity cannot penetrate in the vial. For example, the closing means can be a cork but is preferably a stopper in rubber of pharmaceutical grade, or comprising a rubber part, or an injectable membrane. Such vials and closing means are commonly used to store chemical products, as for example those commercialized by WHEATON® under reference WHEAW224100. The rubber has a good air and water tightness and ensures that the tissue matrix packaged in the vial remains dry and under vacuum. While a rubber stopper can be put in place by pressing it inside the vial opening, an impermeable membrane can be sealed to a vial by a thermal process or using an adhesive.

Advantageously, the closing means is suitable for reversible needle perforation. This means that a needle can be inserted through the closing means to create a fluid communication between the inside and the outside of the vial and that, after removal of the needle, the airtight and watertight features of the stopper are restored. Typically, the needle has one free extremity and the other extremity is connected to a syringe. Stoppers in rubber or injectable membranes typically allow reversible perforation, but any other closing allowing such features can be envisaged.

The closing means can be further secured to the vial, inside or outside the lyophilizer, with additional securing means like, for example, a metal ring or a plastic screw ring, in order to make the seal stronger and confer it an even longer shelf life.

Preferably, the method enables to have a packaged tissue matrix having a rate of humidity (or water content) below 10% by weight and preferably below 6% by weight, measured from the weight difference of the tissue before and after submission to heat.

A closing means such a rubber stopper or injectable membrane present the additional advantage that a syringe needle can be introduced through it without breaking the air and water tightness. This is of particular interest for the introduction of regeneration fluid on the tissue matrix prior to its use for grafting.

The vial with its closing means also ensure that contamination of the matrix tissue is avoided during storage.

The size of the vial is adapted to the size of the piece of tissue matrix it is intended to contain, as well as the amount of regenerating fluid that is necessary to regenerate the tissue.

The invention will be better understood with the following description of several examples, referring to the accompanying drawing on which:

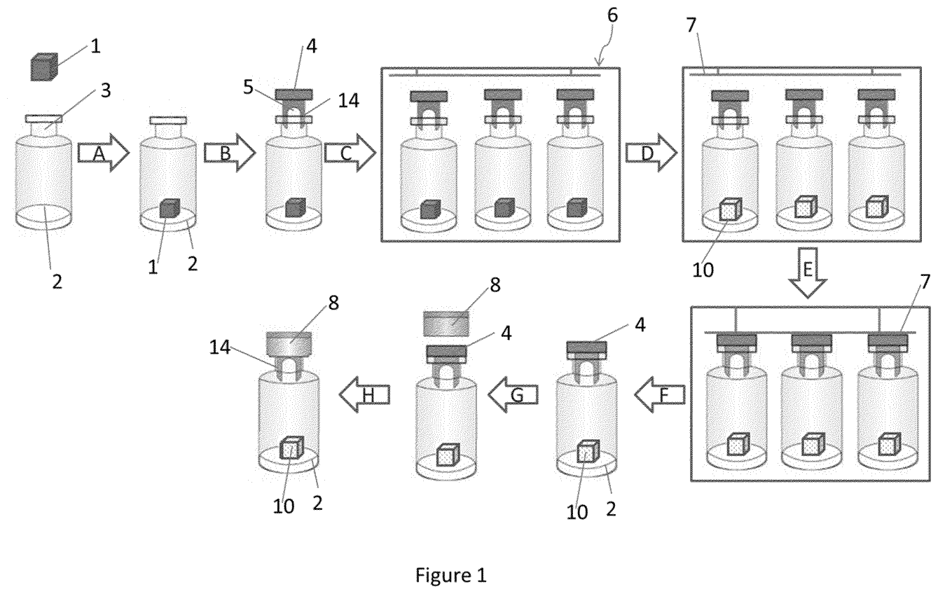

FIG. 1 illustrates the method of the invention.

Referring to FIG. 1, an ex-vivo tissue sample, which originates from an biological tissue having undergone several cleaning steps, manually, for example as disclosed in Biomaterials 23 (2002) 2979-2988, or in an automated manner, as disclosed in PCT/EP2017/083540, is introduced in a vial 2 in a step A. The ex-vivo tissue sample 1 can be here, for example, a piece of bone matrix, and the vial 2 is here a cylindrical glass vial, with a narrower neck 3.

In a step B, a stopper 4, here in rubber, is positioned partially inserted in the neck 3 of the vial 2, in such a way that an opening 5 remains for fluid communication between the inside and the outside of the vial.

This step B of pre-positioning the stopper on the opening of the vial is particularly adapted for the kind of stopper 4 illustrated, having legs 14 enabling the stopper to be blocked in the neck of the vial while leaving an open space 5 between the legs 14 to enable circulation of gas and humidity between the ex-vivo tissue sample 1 and the outside of the vial 2.

However, there are other means to insert the stopper in the neck of the vial at later stages of the method, as will become apparent below.

In step C, the vial 2 containing the ex-vivo tissue sample 1 and topped with stopper 4 in semi-open position in placed in the lyophilizer 6. A simple lyophilizer containing three vials is here illustrated for simplicity, but a lyophilizer can usually contain several vials, even several hundreds of vials, depending on their size, laid out on one or several shelves designed to accommodate vials in a way that they cannot freely move around on the shelf (for example with a rack), or under other arrangements depending on the configuration of the lyophilizer.

When all vials to be lyophilized have been introduced in the lyophilizer, the lyophilizer is closed and the lyophilization process can be performed in step D. Typically, the temperature inside the lyophilizer is reduced to freeze the content of the vials, here the ex-vivo tissue sample 1, and the pressure is reduced to the point of sublimation of the water content of the ex-vivo tissue sample 1.

The process is continued to obtain the tissue matrix 10, as dry as possible, preferably a water content below 10% by weight and still preferably of 6% by weight or less.

At the end of the lyophilization process, in step E, before the lyophilizer is open and the vacuum breached, the stoppers 4 are pressed upon for their complete insertion into the neck 3 of the vial. That way, the tissue matrix 10, almost completely dehydrated, is isolated from the outside of the vial, under vacuum.

In practice, an actionable plate 7 can be installed inside the lyophilizer, above the vials. At the end of the lyophilization process, the plate 7 can be actioned downward in order to exert a pressure on the pre-positioned stopper(s) 4, until the stoppers are completely inserted in the neck 3 of the vial(s) 2.

Alternatively, the stopper(s) can be initially attached/adhered to the plate 7 and be inserted fixedly in the neck 3 of the vials 2 upon a downward pression. It could also be envisaged, when an array of vials is arranged on a shelf of a lyophilizer, that the plate or a roll arranged in the lyophilizer deposits a film on the vials, and that the film is thermo-sealed to the top collar of the neck, by application of heat coming, for example from the plate or the same roll, which could be heatable.

The person skilled in the art thereby understands that there are several technical manners to implement a step of sealing the vials inside a lyophilizer.

In step F, the lyophilizer is open and the vial 2, sealed, and comprising a dehydrated biological tissue matrix 10 under vacuum, to be regenerated for grafting, is removed.

The matrix 10 is now packaged in a form that will allow a long shelf life and an easy use. The vacuum inside the vial will have a tendency to exert a suction onto the rubber stopper, thereby helping maintaining it in place. Because the tissue matrix is not exposed to ambient air after lyophilization, the humidity content of the packaged material remains below 10% by weight and preferably below 6% by weight, allowing a much longer shelf—life of the tissue matrix compared to existing matrixes currently on the market.

Optionally, to reinforce the sealing of the vial, in particular for rubber stoppers and glass vials, an aluminum cap 8 can be placed on the sealed vial 2 in step G, and the cap can be crimped onto the vial neck 3 in step F, in order to fasten the cap 8 and rubber stopper 4 to the vial and avoid any unwanted opening.

Advantageously the cap does not fully cover the top of the stopper or comprises an openable strip to easily uncover an area of the top of the rubber stopper. This present some advantages for further use of the tissue matrix in the vial.

Though a single cubic piece of bone matrix 10 is here illustrated in the vial 2, the invention is not limited to any particular tissue matrix to be regenerated, having any kind of shape. It could for example be other tissue matrixes like skin, dermis, tendon, cortical bone powder, bladder, skeletal muscle . . . .

There may be, in certain cases more than one piece of tissue matrix per vial.

A step of labeling of the vial can be foreseen, in case labeling was not performed before. The label typically contains an identifier (barcode, QR code, RFID chip . . . ) for traceability of the tissue matrix, as is well known in the field.

The tissue matrix can have been prepared through cleaning steps performed by an operator in a sterile facility, in a completely manual way or using some automated steps. The tissue matrix can also have been prepared in a completely automated manner, like for example in a reactor as described in PCT/EP2017/083540 which comprises:
an airlock entry for introduction and classification (introduction into sterile area responding to regulatory standards) of a biological tissue;
means to treat the biological tissue arranged to produce a tissue matrix;
means for packaging the tissue matrix;
an airlock exit of the packaged tissue matrix;
automated means to move the biological tissue from the airlock entry to the treatment means, and
automated means to move the biological tissue matrix from the treatments means to the packaging means and further to the airlock exit.

In such a case, the method of the invention is related to the packaging means as well as the treatment means (lyophilisation).

The method to regenerate the biological tissue matrix 10, packaged in the vial 2 sealed under vacuum, comprises introducing in the vial a regenerating fluid to regenerate the tissue matrix to a biological tissue. After the tissue is regenerated, it will suitable to be grafted to a patient.

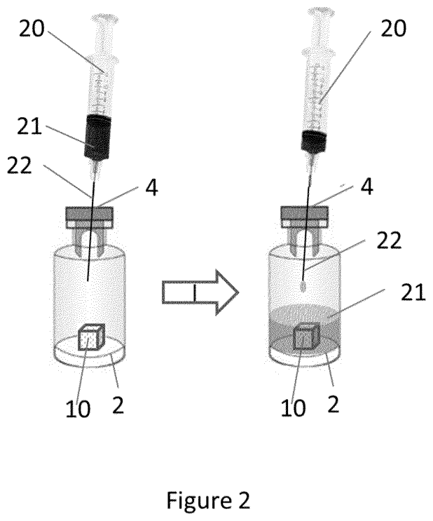

Referring to FIG. 2, the step I of introducing in the vial a regenerating fluid to regenerate the tissue matrix 10 to a biological tissue can be performed through the use of a syringe initially containing the regenerating fluid 21. The free extremity of the needle 22 of the syringe 20 is introduced in the vial 2 through the stopper 4, which can be a rubber stopper or an injectable membrane. Through pressing on the piston of the syringe and/or suction force resulting from the pressure difference between the inside of the vial and the external environment, the regeneration fluid is transferred into the vial, onto the tissue matrix 10. The needle of the syringe is then removed from the stopper which re-seals, leaving no opening (hence the reversible perforation of the stopper by the needle).

After a period of time, which can depend on the type of tissue, the matrix is regenerated to a biological tissue, suitable to be grafted to a patient. The professional merely needs to open the vial and sample the tissue.

The regenerating fluid can be cells in suspension in blood, plasma rich platelet, bone marrow, lipoaspirate, a growth factors solution, a culture media with stem cells or a suspension of a bioactive agent . . . .

The advantage of using the tissue matrix in the vial as described above is that sterility of the tissue can be ensured up to the last minute before grafting, as the number of manipulation step of the tissue is reduced, the tissue is not put in contact with other containers and the vial remains has closed to any potential contamination.

Alternatively, in case the stopper is not suitable for a needle perforation, for example when the tissue matrix is packaged under vacuum in a plastic tray sealed with a plastic film as closing means, the film is removed, regeneration fluid is added in the vial. Time is allowed for regeneration of the tissue matrix to a biological tissue suitable for grafting. These steps can be performed directly in the operating theatre, where contamination is highly controlled.

The regenerating fluid can originate from the patient himself and can for example be cells in suspension in blood, plasma rich platelet, bone marrow, lipoaspirate, a growth factors solution, a culture media with stem cells or any other bioactive agents. The tissue matrix can originate from the patient himself and can, for example, be bone, skin, dermis, tendon, cortical bone powder, bladder, skeletal muscle.

A patient can be a human patient or an animal.

1. A method for producing a vial, under vacuum, including a biological tissue matrix to be regenerated for grafting, the method comprising:
placing a treated ex-vivo tissue sample in an open rigid vial;
placing the vial in a lyophilizer;
running a lyophilization process under vacuum to convert the treated ex-vivo tissue sample into a biological tissue matrix;
hermetically sealing the vial with closing means, inside the lyophilizer under vacuum, and
removing the sealed vial from the lyophilizer.

2. The method according to claim 1, further comprising, before running the lyophilization process:
pre-positioning the closing means onto the vial in a manner enabling fluid communication through the opening of the vial.

3. The method according to claim 1, wherein hermetically sealing the vial with the closing means comprises applying a pressure onto the closing means.

4. The method according to claim 1, performed under sterile conditions.

5. A vial sealed under vacuum with closing means and comprising a biological tissue matrix.

6. The vial according to claim 5, wherein the closing means comprise a rubber stopper or an injectable membrane.

7. The vial according to claim 5, wherein the closing means is suitable for reversible needle perforation.

8. The vial according to claim 5, wherein the vial is made of glass or rigid polymer.

9. The vial according to claim 5 wherein the tissue matrix has a rate of humidity below 10% by weight, preferably below 6% by weight.

10. The vial according to claim 5, wherein the tissue matrix is sterile and is for pharmaceutical use, clinical use or veterinary use.

11. A use of the vial of claim 4 for producing a regenerated tissue from the biological tissue matrix.

12. The method to regenerate a biological tissue matrix packaged in a vial according to claim 5, comprising the steps of:
introducing in the vial a regenerating fluid to regenerate the tissue matrix to a biological tissue.

13. The method according to claim 10, wherein introducing the regenerating fluid in the vial comprises injecting, inside the vial, regenerating fluid contained in a syringe by:
reversibly perforating the opening means of the vial with the needle of the syringe;
transferring the regenerating fluid from the syringe to the vial, and withdrawing the needle of the syringe from the opening means.

14. The method according to claim 10, wherein the regenerating fluid comprises cells in suspension in blood, plasma rich platelet, bone marrow, lipoaspirate, a growth factors solution, a culture media with stem cells or a suspension of a bioactive agent.

15. The method according to claim 1, wherein the tissue matrix originates from one of:
bone,
skin,
dermis,
tendon,
cortical bone powder,
bladder, and
skeletal muscle
